(19) United States
(12) Patent Application Publication
(10) Pub. No.: US 2021/0236047 A1
(43) Pub. Date: Aug. 5, 2021

(54) FETAL MOVEMENT DETECTION DEVICE AND FETAL MOVEMENT DETECTION SYSTEM

(71) Applicant: ProtoMarketing Inc., Takatsuki-shi, Osaka (JP)

(72) Inventor: Yuya YASUTAKE, Takatsuki-shi (JP)

(21) Appl. No.: 17/270,157

(22) PCT Filed: Nov. 18, 2019
(86) PCT No.: PCT/JP2019/045140

§ 371 Date: Feb. 22, 2021

(30) Foreign Application Priority Data
Dec. 3, 2018 (JP) 2018-226200

(51) Int. Cl. A61B 5/00; A61B 5/11

(52) U.S. Cl. CPC A61B 5/4362; A61B 5/0022; A61B 2562/164; A61B 5/6823; A61B 5/1126

(57) ABSTRACT

Provided are a fetal movement detection device and a fetal movement detection system for home use. The fetal movement detection device includes a sheet body, a plurality of sensors, and a controller. The sheet body which is bendable and extends planarly is comprised of a first sheet layer and a second sheet layer which are fixed to each other, and a back surface of the sheet body contacts an abdomen of a maternal body of a pregnant person. Each sensor has a sensor body to detect a fetal movement of the fetus inside a uterus of the maternal body, and an output line connected to an output end of the sensor body. In each sensor, the entire sensor body and an output end of the output line are both covered by the sheet body. The fetal movement detection system includes the device and a cloud server.

### TECHNICAL FIELD

The present disclosure relates to a fetal movement detection device configured to detect movements of a fetus in a uterus of a pregnant maternal body while the pregnant being at home, and relates to a fetal movement detection system including the fetal movement detection device.

### BACKGROUND ART

Various devices are proposed which detect fetal movements and heart sounds of a fetus in a pregnant maternal body to acquire information on the fetal movements and the heart sounds of the fetus (see, for example, Patent Documents 1-8).

### REFERENCE DOCUMENTS OF CONVENTIONAL ART

### Patent Documents

[Patent Document 1] JP2016-531642A

[Patent Document 2] JP2014-002823A1

[Patent Document 3] JP2013-169403A

[Patent Document 4] JP2012-252358A

[Patent Document 5] JP2012-152407A

[Patent Document 6] JP2011-516238A

[Patent Document 7] JP2003-111760A

[Patent Document 8] JP2001-276079A

### DESCRIPTION OF THE DISCLOSURE

### Problem to be Solved by the Disclosure

According to the conventional arts, a pregnant can confirm information on fetal movements and heart sounds of a fetus in the maternal body by the information being detected, and further can transmit the information to a doctor, while being at home etc.

However, it is demanded that the pregnant can utilize the acquired information on the fetal movements etc. of the fetus in a combined manner from various viewpoints.

The present disclosure is made in view of such a demand or problem, and a main purpose thereof is to provide a fetal movement detection device which allows a pregnant to easily and securely acquire various data related to fetal movements of a fetus in the pregnant maternal body, while being at home, by utilizing sensors which can detect the fetal movements of the fetus in a combined manner.

### SUMMARY OF THE DISCLOSURE

A fetal movement detection device according to the subject of the present disclosure includes a bendable sheet body extending planarly and including a back surface to be contacted with an abdomen of a pregnant maternal body, an output end provided to a front surface of the sheet body, a plurality of sensors, each including a sensor body configured to detect a fetal movement of a fetus inside a uterus of the maternal body, and an output line connected to an output end of the sensor body and wired so as to converge on the output end on the front surface of the sheet body, and a controller including an input end electrically connected to the output end of the front surface of the sheet body and configured to output a signal to an external device. The entire sensor body and the entire output line of each of the plurality of sensors are both covered by the sheet body, while the controller is not covered by the sheet body.

### Effect of the Disclosure

According to the fetal movement detection device of the subject of the present disclosure, a pregnant can freely and securely acquire various data related to daily fetal movements of a fetus in the pregnant maternal body, while being in own house or at home.

Hereinafter, various embodiments of the present disclosure will be described together with its effects and advantages based on the accompanying drawings.

### BRIEF DESCRIPTION OF DRAWINGS

FIG. 1 is a view schematically illustrating a configuration of a fetal movement detection system using a fetal movement detection device according to Embodiment 1 of the present disclosure.

FIGS. 2(a) and 2(b) are vertical end views schematically illustrating a condition in which sensors of the fetal movement detection device according to Embodiment 1 are covered.

FIGS. 3(a) to 3(d) are views schematically illustrating various information on fetal movements acquired by the fetal movement detection system of the present disclosure.

FIGS. 4(a) and 4(b) are views schematically illustrating information on the fetal movements of the fetus acquired by the fetal movement detection system of the present disclosure and information on heart sounds of the fetus acquired by a microphone.

FIG. 5 is a view schematically illustrating a configuration of a fetal movement detection system provided with a fetal movement detection device according to Embodiment 2 of the present disclosure.

FIGS. 6(a) and 6(b) are vertical end views schematically illustrating a condition in which sensors of the fetal movement detection device according to Embodiment 2 are covered.

FIG. 7 is a front view schematically illustrating a configuration of a modification of the fetal movement detection device according to Embodiment 1 or 2.

### MODES FOR CARRYING OUT THE DISCLOSURE

### Embodiment 1

<Configurations of Fetal Movement Detection System and Fetal Movement Detection Device>

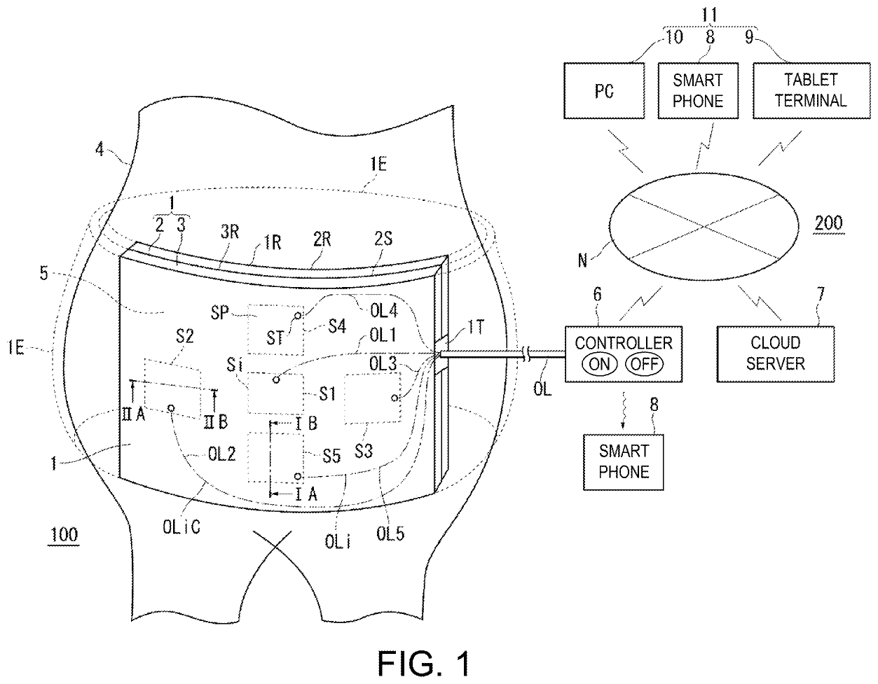

FIG. 1 is a view schematically illustrating a configuration of a fetal movement detection system 200 according to this embodiment. In FIG. 1, the fetal movement detection system 200 has a fetal movement detection device 100 as a main component. This fetal movement detection device 100 is a device which allows a pregnant to easily and securely know fetal movements of a fetus (not illustrated) inside a uterus of the pregnant maternal body, i.e., a condition of physical movements of each part (hands, feet, or head) of the fetus inside the uterus, while being at home, by a plurality of sensors detecting the fetal movements. For this purpose, the fetal movement detection device 100 is generally comprised of (1) a sheet body 1, (2) an output-line part OL comprised of a bunch of output lines and electrically connected to an output end 1T of the sheet body 1, and (3) a controller 6 as a control device, having an input end (not illustrated) electrically connected to each of the output lines of the output-line part OL. Moreover, the fetal movement detection system 200 includes a cloud server 7 electrically connected with the fetal movement detection device 100 or the controller 6 through communication with the Internet N, and a terminal device 11 (e.g., a smartphone 8, a tablet terminal 9, or a personal computer (PC) 10) which carries out intercommunication with the cloud server 7 through the Internet N. Below, a detailed configuration of the fetal movement detection device 100 will be described.

The sheet body 1 is a planarly extending sheet which entirely has flexibility so that it can be arranged on an abdomen 5 of a pregnant maternal body 4 while it can freely bend along the abdomen 5. In the example in FIG. 1, the sheet body 1 has a substantially rectangular parallelepiped shape of a given size which can cover the abdomen 5. Thus, as illustrated in FIG. 1, a back surface 1R of the sheet body 1 entirely contacts the abdomen 5 of the maternal body 4. The contact condition between the back surface 1R of the sheet body 1 and the skin surface of the abdomen 5 illustrated in FIG. 1 may be achieved, for example, by an adsorptive sheet or an adsorptive disk (not illustrated) which is provided in advance on the back surface 1R of the sheet body 1, or by hands of the pregnant or a third person (e.g., her husband) holding the sheet body 1 so as not to slip down. In any case, the sheet body 1 is used with its back surface 1R entirely contacted with the abdomen 5 of the pregnant maternal body 4 over the entire surface.

Moreover, in this embodiment, as illustrated in FIG. 1, the sheet body 1 is constituted as a “bilayer sheet” comprised of a first sheet layer 2 which extends in a longitudinal direction, and a second sheet layer 3 which is provided to be laminated on the first sheet layer 2 on its entire surface. That is, the first sheet layer 2 is a bendable sheet extending planarly in the longitudinal direction, which has a back surface 2R corresponding to the back surface 1R of the sheet body 1, and a front surface 2S opposing the back surface 2R. On the other hand, the second sheet layer 3 has a back surface 3R connected or fixedly provided to the front surface 2S of the first sheet layer 2 over its substantially entire surface, and is a laminated sheet which is bendable and extends planarly in the longitudinal direction similarly to the first sheet layer 2. Here, the first sheet layer 2 and the second sheet layer 3 are made of a fabric-like material, a mesh-like material, or paper. For example, if a commercially available fabric-like material which has a function of blocking electromagnetic wave is used as the first sheet layer 2 and the second sheet layer 3, the sheet body 1 exhibits a function of preventing propagation of the electromagnetic wave on the pregnant.

Moreover, in this embodiment, as an important component of the fetal movement detection device 100, a plurality of sensors Si (i: 1, 2, 3, 4, 5) are provided vertically and horizontally inside the sheet body 1. In the example illustrated in FIG. 1, five sensors are provided vertically and horizontally on the same surface. For convenience of illustration, the sensors Si (i: 1, 2, 3, 4, 5) are each illustrated schematically by a broken line. Here, the concept of “a plurality of sensors Si” means that the number of sensors is at least two and that an arbitrary number of sensors are provided. The plurality of sensors Si (i: 1, 2, 3, 4, 5) each has 1) a sensor body SP to detect a fetal movement of a fetus inside the uterus of the maternal body 4, and 2) an output line OLi (i: 1, 2, 3, 4, 5) connected to an output end ST of the sensor body SP. Among them, the sensor body SP of each sensor Si (i: 1, 2, 3, 4, 5) has a flexible substrate (not illustrated) which is bendable according to a degree of bending of the sheet body 1 when the sheet body 1 contacts the abdomen 5 of the maternal body 4. On this substrate, a detection part (not illustrated) is formed. On the other hand, the output line OLi (i: 1, 2, 3, 4, 5) of each sensor Si (i: 1, 2, 3, 4, 5) is wired inside the sheet body 1, as illustrated in FIG. 1, so as to converge on the output end 1T which is provided to one end surface of the sheet body 1 in a short direction, and is then further wired from the output end 1T toward the controller 6 outside, as one of the output lines of the output line part OL (already described).

As each sensor Si (i: 1, 2, 3, 4, 5), a pressure sensor is used, for example. Alternatively, an acceleration sensor is used as each sensor Si (i: 1, 2, 3, 4, 5). Alternatively, a Doppler sensor is used as each sensor Si (i: 1, 2, 3, 4, 5).

Alternatively, instead of using only one type of sensor (with the same configuration and the same function) as each sensor Si (i: 1, 2, 3, 4, 5) as described above, a plurality of types of sensors with configurations and functions different from each other may be used as sensors Si (i: 1, 2, 3, 4, 5). In this case, various information on the fetal movements of the fetus is acquired by different types of sensors, which allows for utilization of the information or data in a combined manner, as will be described below. For example, a plurality of pressure sensors (corresponding to a “first sensor”) and a plurality of acceleration sensors (corresponding to a “second sensor”) may be used as the plurality of sensors Si (i: 1, 2, 3, 4, 5). Alternatively, the plurality of sensors Si (i: 1, 2, 3, 4, 5) may be comprised of a plurality of pressure sensors (corresponding to the “first sensor”) and a plurality of Doppler sensors (corresponding to the “second sensor”), or comprised of a plurality of acceleration sensors (corresponding to the “first sensor”) and a plurality of Doppler sensors (corresponding to the “second sensor”), respectively. Alternatively, the plurality of sensors Si (i: 1, 2, 3, 4, 5, . . . ) may be comprised of a plurality of pressure sensors (corresponding to the “first sensor”), a plurality of acceleration sensors (corresponding to the “second sensor”), and a plurality of Doppler sensors (corresponding to a “third sensor”).

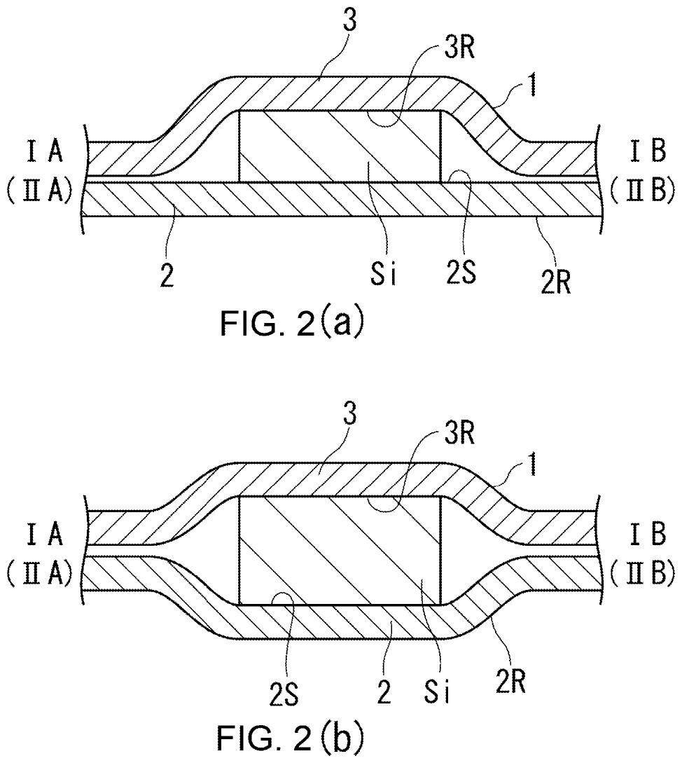

Here, a remarkable feature is that, in each of the plurality of sensors Si (i: 1, 2, 3, 4, 5), the entire sensor body SP and the entire of a part OLiC (a part illustrated by a one-dot chain line in FIG. 1) of the output line OLi (i: 1, 2, 3, 4, 5) at the output end ST side are both covered by the sheet body 1. Describing based on this embodiment, in each of the plurality of sensors Si (i: 1, 2, 3, 4, 5), the entire sensor body SP and the entire of the part OLiC (the part illustrated by the one-dot chain line in FIG. 1) of the output line OLi (i: 1, 2, 3, 4, 5) at the output end ST side are both covered so that they are wrapped by the front surface 2S of the first sheet layer 2 and the back surface 3R of the second sheet layer 3. FIGS. 2(a) and 2(b) illustrate vertical end views schematically illustrating this feature along cutting lines IA-IB and IIA-IIB in FIG. 1. Among FIGS. 2(a) and 2(b), FIG. 2(a) is a vertical end view illustrating the covering condition of the sensor Si when the sensor Si is a “pressure sensor” having a weight relatively smaller than that of other types of sensor. In this case, both a part of the front surface 2S of the first sheet layer 2, which contacts a lower surface of the sensor Si, and a part of the back surface 2R, which opposes the contact part of the front surface 2S, form a planar surface, and the first sheet layer 2 is not depressed in a concave shape. On the other hand, FIG. 2(b) is a vertical end view illustrating the covering condition of the sensor Si when the sensor Si is an “acceleration sensor” or a “Doppler sensor” which has a weight relatively larger than that of the “pressure sensor.” In this case, the part of the first sheet layer 2 contacting the sensor Si and a peripheral part of the contact part are depressed in a concave shape, and a planar-surface part opposing the sensor Si in the back surface 2R of the first sheet layer 2 contacts the abdomen 5 of the maternal body 4.

According to such a configuration of the feature, in each of the plurality of sensors Si (i: 1, 2, 3, 4, 5), the entire sensor body SP and the entire of the part OLiC (the part illustrated by the one-dot chain line in FIG. 1) of the output line OLi (i: 1, 2, 3, 4, 5) at the output end ST side are protected from the outside by the sheet body 1. Thus, it is prevented that the pregnant or other person, or an outside object directly contacts the sensor body SP etc. of each sensor Si (i: 1, 2, 3, 4, 5), and, as a result, it is prevented that an electric failure (e.g., short circuit of each sensor Si) or a mechanical failure occurs. Moreover, the pregnant is not required to perform operations of measuring and detecting fetal movements of the fetus in a multidimensional manner by applying the sensors Si (i: 1, 2, 3, 4, 5) to the surface of her own abdomen 5, while directly holding the sensors Si. The pregnant just keeps the sheet body 1 in contact with her own abdomen 5 during the detection of the fetal movements of the fetus.

<Operations and Advantages of Fetal Movement Detection System>

In FIG. 1, when the pregnant brings the back surface 1R of the sheet body 1 to entirely contact with the curved shape of the abdomen 5, and then sets the controller (control device) 6 electrically connected to the sheet body 1 from an “OFF” state to an “ON” state, the fetal movement detection system 200 performs the following operations. That is, the controller 6 receives a signal detected by each sensor Si provided inside the sheet body 1 according to the fetal movement status of the fetus, which changes momentarily, through the corresponding output line OLi. Moreover, the controller 6 transmits the output signal which is momentarily sent from each sensor Si described above to the cloud server 7, through a communication via the Internet N (e.g., communication via WiFi®). Then, the cloud server 7 receives the output signal of each sensor Si described above, and processes information provided by the output signal described above based on an application software program held by the cloud server 7. Through such an information processing, the cloud server 7 calculates and acquires various information (various data) of the fetal movements of the fetus (i.e., the data from each sensor Si is utilized), and holds these data in a database (not illustrated). For example, the cloud server 7 calculates and acquires information about in what position inside the uterus the fetus is located, or in what direction the fetus's feet are oriented inside the uterus, when the data is acquired from the controller 6. In addition, if the terminal device 11 (e.g., the PC 10, the smartphone 8, or the tablet terminal 9) operated by the pregnant or her husband or family (hereinafter, referred to as “pregnant etc.”) accesses the cloud server 7 via the Internet N, the cloud server 7 displays the calculated and held various data related to the fetal movements of the fetus on a display (not illustrated) of the terminal device 11 via the Internet N. Accordingly, the pregnant etc. can visually recognize the various data related to the fetal movements of the fetus, which is displayed on the display of the terminal device 11 described above, or can hold the data in a memory medium inside the terminal device 11. Therefore, the pregnant etc. can freely and securely grasp and know any time the condition or status of the own fetus inside the maternal body 4, which changes momentarily, while being in own house or at home.

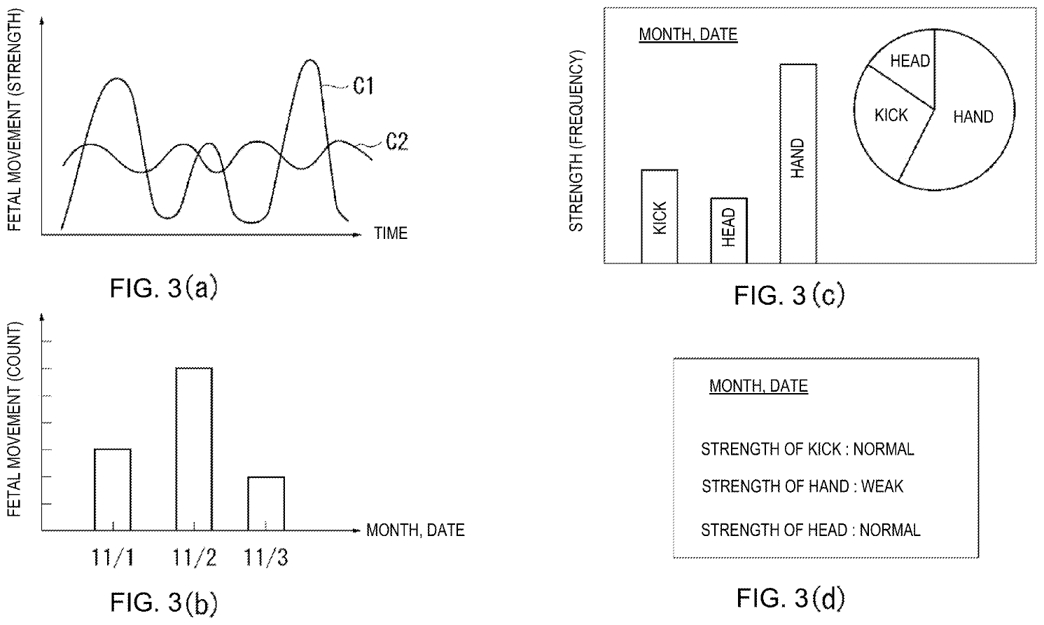

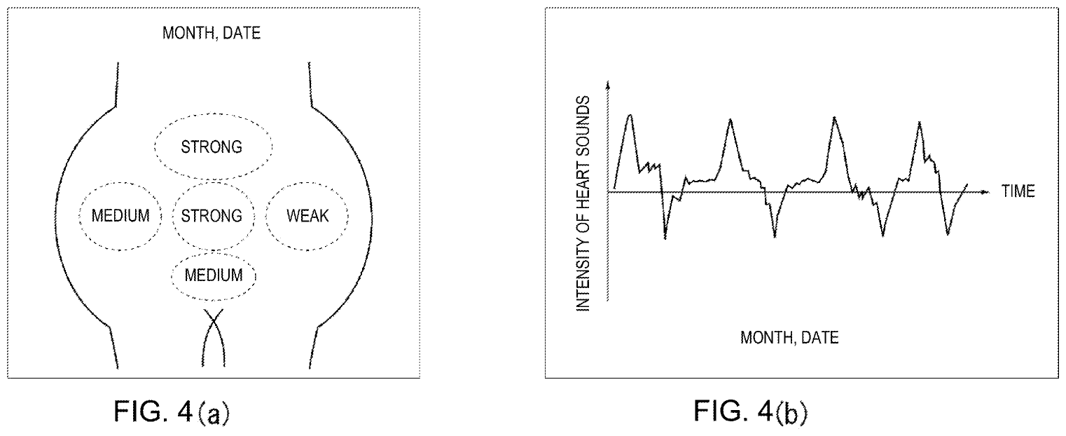

FIGS. 3(a), 3(b), 3(c), 3(d), and 4(a) schematically illustrate the various data related to the fetal movements of the fetus, which are given to the pregnant etc. by such a cloud server 7.

For example, as illustrated in FIG. 3(a), the cloud server 7 displays for the pregnant etc. a variation of a strength of the fetal movements of the fetus over time in the form of a curve C1, according to the acquisition and calculation results of the detection signal of each sensor Si and an instruction from the terminal device 11. Alternatively, the cloud server 7 displays a “hiccup” movement of the fetus inside the uterus in the form of a sinewave-like curve C2, based on the detection signal from the pressure sensor Si. Note that, in the latter case, it is supposed that the “hiccup” movement described above is detected by the pressure sensor Si as a movement of the “head” part of the fetus.

Alternatively, as illustrated in FIG. 3(b), the cloud server 7 displays for the pregnant etc. a frequency or the number of the fetal movements of the fetus of some days or everyday in a time series, in the form of a bar graph, according to the acquisition and calculation results of the detection signal of each sensor Si and the instruction from the terminal device 11.

Alternatively, as illustrated in FIG. 3(c), the cloud server 7 displays for the pregnant etc. the strength (frequency) of the movement of each of the fetus's parts (i.e., feet (kick), hands, and head) for a specific date, in the form of a bar graph together with a circle graph, according to the acquisition and calculation results of the detection signal of each sensor Si and the instruction from the terminal device 11, so that the determination result is more visually understandable.

Alternatively, as illustrated in FIG. 3(d), the cloud server 7 displays results of evaluation of the strength (“normal,” “strong,” or “weak”) of the movement of each of the fetus's parts (i.e., feet, hands, and head) for a specific date, based on a comparison with an average calculated from the fetal movement data which are accumulated daily.

Alternatively, if the sensors of the same type (e.g., pressure sensors) are provided as the sensors Si of FIG. 1, as illustrated in FIG. 4(a), the cloud server 7 displays for the pregnant etc. the result of determination on the strength of the fetal movements of the fetus at each position inside the pregnant's abdomen, for a specific date or time zone, in the form of a distribution chart of “strong,” “medium,” and “weak.”

Alternatively, if the cloud server 7 is to acquire, calculate, and hold various data of fetal movements of a fetus for many pregnants, the fetal movement detection system 200 also exerts advantages as described below. That is, each pregnant can relatively make a determination for various information related to the fetal movements of her own fetus, based on a comparison with data of fetal movements of other pregnant's fetus.

For example, through the operations of the fetal movement detection system 200, each pregnant can visually know a degree of influence of her daily food intake on a state of the fetus, via the terminal device 11 such as the smartphone 9, and can also leave the information stored in the terminal device 11. For example, depending on the intake of a specific food (e.g., rice, wheat, or eggs) by the pregnant, if a food allergy reaction of the fetus is suspected from the data of the condition of the fetus, the pregnant can suppress in advance the occurrence of the food allergy of the fetus, by avoiding excessive intake of the food from then.

As described above, the fetal movement detection device 100 and the fetal movement detection system 200 using the device 100 provide utilization of the functions of the sensors Si which are capable of detecting the fetal movements of the fetus inside the pregnant maternal body 4 in a combined manner, and thus allow the pregnant to freely, easily, and securely acquire various data related to the fetal movements of the fetus from multiple viewpoints, while being at home, only by placing or wrapping the planar sheet body 1 around her own abdomen 5.

### Embodiment 2

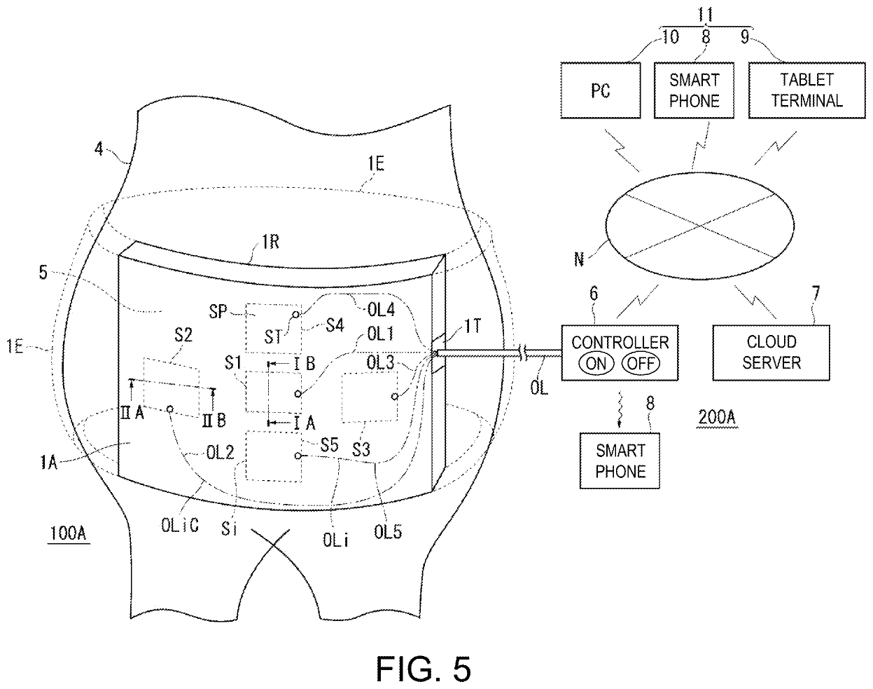

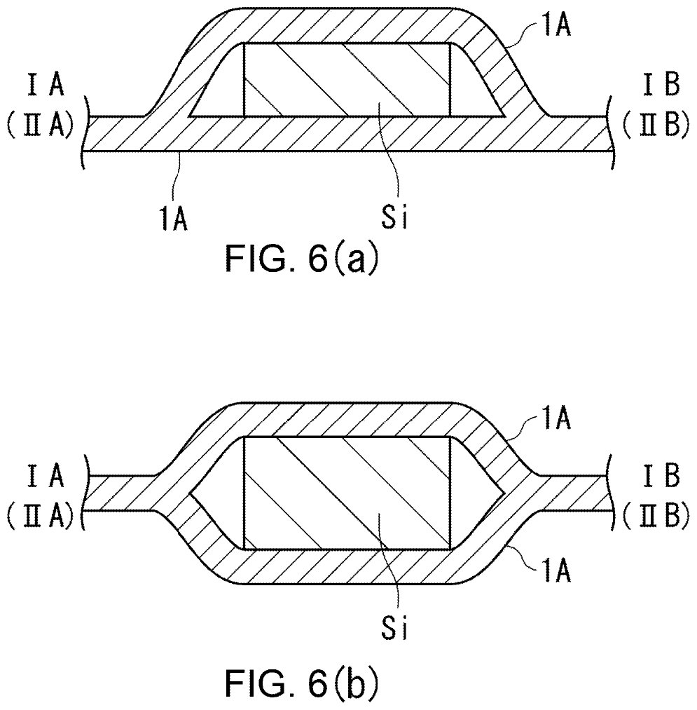

FIG. 5 is a view schematically illustrating a configuration of a fetal movement detection system 200A using a fetal movement detection device 100A according to this embodiment. As illustrated in FIG. 5, a feature of the fetal movement detection device 100A is that a sheet body 1A, an alternative to the bilayer sheet body 1 in FIG. 1, is comprised of a single layer of synthetic resin as a sheet layer which extends planarly and has flexibility so that it can freely bend along the abdomen 5. For example, the sheet body 1A is made of silicone resin. Moreover, similarly to the sheet body 1, the sheet body 1A has a plurality of sensors Si inside thereof. That is, in each sensor Si, the entire sensor body SP and the entire of the part OLiC of the output line OLi at the output end ST side are both covered by the single synthetic resin layer described above. According to such a configuration, vertical end views schematically illustrated along cutting lines and IIA-IIB in FIG. 5 are as illustrated in FIGS. 6(a) and 6(b), respectively. Among FIGS. 6(a) and 6(b), FIG. 6(a) is a vertical end view illustrating the covering condition of the sensor Si when the sensor Si is a “pressure sensor.” On the other hand, FIG. 6(b) is a vertical end view illustrating the covering condition of the sensor Si when the sensor Si is an “acceleration sensor” or a “Doppler sensor.” Other components of the fetal movement detection device 100A are the same as the corresponding components of the fetal movement detection device 100 in FIG. 1. Moreover, the fetal movement detection system 200A illustrated in FIG. 5 also has a similar configuration to the fetal movement detection system 200 of FIG. 1. Accordingly, the fetal movement detection device 100A and the fetal movement detection system 200A attain the same operations and advantages as already described for Embodiment 1. In this regard, the corresponding description for Embodiment 1 is incorporated.

### (Modifications)

(1) As illustrated in FIGS. 1 and 5, the controller 6 may output the data related to the fetal movements of the fetus, which is acquired from each sensor Si, directly to the smartphone 8 through a communication using Bluetooth® so that the smartphone 8 displays and/or stores the received data described above.

(2) Although in Embodiments 1 and 2 the sheet bodies 1 and 1A have, inside thereof, only the plurality of sensors Si to detect the fetal movements of the fetus, the sheet bodies 1 and 1A may further have, inside thereof, at least one microphone (not illustrated) entirely covered by the sheet body 1 and 1A. In such a case, the cloud server 7 may generate and hold, as heart sound information of the fetus, waveform data indicating a temporal change in an intensity of the heart sounds as illustrated in FIG. 4(b), based on the heart sound data of the fetus acquired as an output signal of the microphone. Alternatively, the cloud server 7 may store and hold the output signal of the microphone described above as sound data. As a result, the pregnant etc. can acquire the waveform data of the heart sounds illustrated in FIG. 4(b) into the terminal device 11, via the Internet N, to visually recognize the temporal change in the heart sounds, or can also acquire the recorded sound data of the fetus's heart sounds into the terminal device 11 to hear the heart sounds.

(3) As illustrated by broken lines 1E in FIGS. 1 and 5, the sheet bodies 1 and 1A may be configured in a bellyband-like shape by being extended so as to wrap one around a waist part of the maternal body 4. The sheet bodies 1 and 1A in such a bellyband-like form are also defined in the present disclosure as “a bendable sheet body extending planarly and including a back surface to be contacted with an abdomen of a pregnant maternal body.” Of course, the sheet bodies 1 and 1A in the bellyband-like form have a plurality of sensors Si etc. of the same type or different types (already described) inside a part of the sheet bodies 1 and 1A at the side of the abdomen 5 of the maternal body 4. Needless to say, also in this case, the operations and advantages of the fetal movement detection system as already described for Embodiment 1 are attained similarly. In this regard, the corresponding description for Embodiment 1 is also incorporated into this modification.

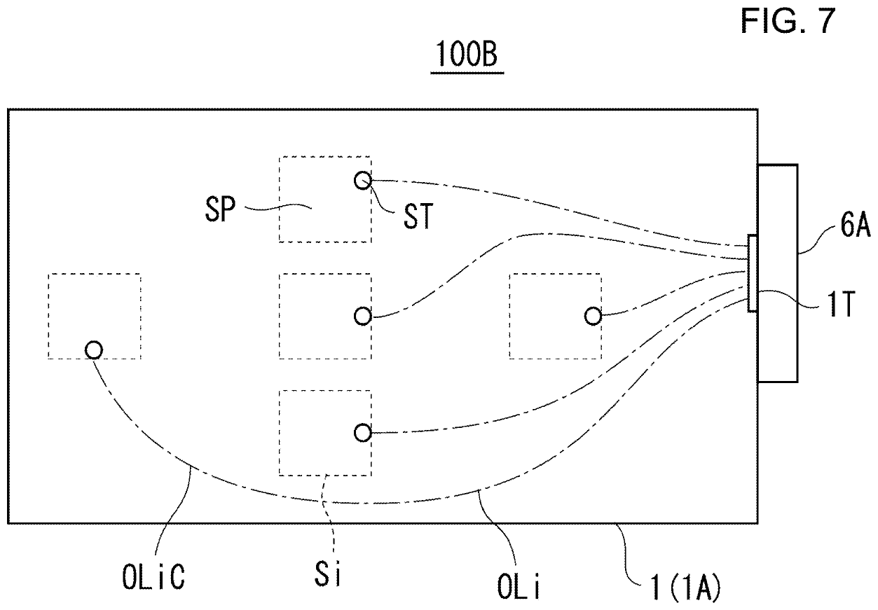

(4) FIG. 7 is a front view schematically illustrating a configuration of a fetal movement detection device 100B according to a modification of the fetal movement detection device 100 or 100A according to Embodiment 1 or 2. Also in FIG. 7, for convenience, each sensor Si and each output line OLiC (OLi) are illustrated by a broken line and a one-dot chain line, respectively, similar to FIGS. 1 and 2. In FIG. 7, a feature of the fetal movement detection device 100B different from that of the fetal movement detection device 100 or 100A is that an input end (not illustrated) of a controller 6A is electrically connected directly to the output end 1T of the sheet body 1 (1A), instead of the controller 6 in FIGS. 1 and 5 being electrically connected to the output end 1T of the sheet body 1 through the output line part OL. In that sense, the controller 6A is electrically connected to the output line OLi of each of the plurality of sensors Si. Other configuration of the fetal movement detection device 100B, the configuration of the fetal movement detection system using the device 100B, and the operations and advantages of the system are the same as those of the fetal movement detection device and the fetal movement detection system as already described for Embodiment 1. Therefore, the descriptions of those for Embodiment 1 are also incorporated into this modification. Note that the “front surface” of the sheet body 1 (1A) where the output end 1T is provided refers to a surface other than the “back surface” of the sheet body 1 (1A) to be contacted with the abdomen of the pregnant maternal body, and corresponds to, for example, each side surface or an upper surface of the sheet body 1 (1A) which is exposed. As illustrated in FIG. 7, the controller 6A which outputs a signal to an external device is integrally provided with the sheet body 1 (1A) on the front surface of the sheet body 1 (1A) described above. However, the controller 6A is not covered by the sheet body 1 (1A), but is exposed.

### (Additional Note)

The embodiments of the present disclosure are disclosed and described in detail as above, but the foregoing description is to illustrate applicable aspects of the present disclosure, and the present disclosure is not limited to this configuration. That is, various changes and/or modifications to the described aspects are conceivable without departing from the scope of the present disclosure.

### INDUSTRIAL APPLICABILITY

For example, the present disclosure is suitably applied to a detection device and a detection system which allow a pregnant to freely know various information on a daily state of a fetus inside a uterus, while being at home.

### DESCRIPTION OF REFERENCE CHARACTERS

200, 200A Fetal Movement Detection System
100, 100A, 100B Fetal Movement Detection Device
N Internet
Si, 51, S2, S3, S4, S5 Sensor
OLi Output Line
OLiC Part of Output Line at Output End Side (Part Covered by Sheet Body)
OL Output Line Part
1 Sheet Body
2 First Sheet Layer
3 Second Sheet Layer
1A Sheet Layer (Single Synthetic Resin Layer)
4 Maternal Body
5 Abdomen
6, 6A Controller (Control Device)
7 Cloud Server
11 Terminal Device

1-4. (canceled)

5. A fetal movement detection device, comprising:
a bendable sheet body extending planarly and including a back surface to be contacted with an abdomen of a pregnant maternal body;
an output end provided to a front surface of the sheet body;
a plurality of sensors, each including a sensor body configured to detect a fetal movement of a fetus inside a uterus of the maternal body, and an output line connected to an output end of the sensor body and wired so as to converge on the output end on the front surface of the sheet body; and
a controller including an input end electrically connected to the output end of the front surface of the sheet body and configured to output a signal to an external device,
wherein the entire sensor body and the entire output line of each of the plurality of sensors are both covered by the sheet body, while the controller is not covered by the sheet body.

6. The fetal movement detection device of claim 5, wherein the plurality of sensors include at least a first sensor and a second sensor having a sensor function different from the first sensor.

7. A fetal movement detection device, comprising:
a bendable sheet body extending planarly and including a back surface to be contacted with an abdomen of a pregnant maternal body;
an output end provided to a front surface of the sheet body;
a plurality of sensors, each including a sensor body configured to detect a fetal movement of a fetus inside a uterus of the maternal body, and an output line connected to an output end of the sensor body and wired so as to converge on the output end on the front surface of the sheet body; and
a controller including an input end directly connected to the output end of the front surface of the sheet body, provided to the front surface of the sheet body integrally with the sheet body, and configured to output a signal to an external device,
wherein the entire sensor body and the entire output line of each of the plurality of sensors are both covered by the sheet body, while the controller is not covered by the sheet body.

8. The fetal movement detection device of claim 7, wherein the plurality of sensors include at least a first sensor and a second sensor having a sensor function different from the first sensor.

9. A fetal movement detection system, comprising:
the fetal movement detection device of claim 5; and
a cloud server configured to receive a signal outputted from the controller via the Internet, calculate and hold data related to the fetal movement of the fetus based on the output signal of the controller, and transmit, via the Internet, the data related to the fetal movement to a terminal device accessing via the Internet.
